(19) United States
(12) Patent Application Publication
(10) Pub. No.: US 2024/0138892 A1
(43) Pub. Date: May 2, 2024

(54) SEGMENTED BONE CEMENT EXTRACTOR, AND METHOD FOR EXTRACTING CEMENT FROM BONE

(71) Applicant: Shukla Medical, St. Petersburg, FL (US)

(72) Inventor: Austin Ryan Stroh, Lutz, FL (US)

(73) Assignee: Shukla Medical, St. Petersburg, FL (US)

(21) Appl. No.: 18/495,914

(22) Filed: Oct. 27, 2023

Related U.S. Application Data
(60) Provisional application No. 63/381,714, filed on Oct. 31, 2022.

(51) Int. Cl. A61B 17/88

(52) U.S. Cl. CPC A61B 17/8847; A61B 2017/0046

(57) ABSTRACT

A bone cement extractor for extracting cement from a bone cavity comprising a connection body and a segmented assembly. The connection body has a quick connect for connecting to a working tool. The segmented assembly is attachable to a distal end of the connection body and includes an inner segment having a proximal end and a distal end, the proximal end connectable to the distal end of the connection body, and an outer segment having a proximal end and a distal end, the outer segment slidably connectable to the inner segment.

### CROSS-REFERENCE TO RELATED APPLICATIONS

This application claims the benefit under 35 U.S.C. § 119(e) to U.S. Provisional Patent Application No. 63/381,714, filed Oct. 31, 2022, the entire disclosure of which is hereby incorporated by reference for all purposes.

### FIELD OF THE INVENTION

The present invention relates generally to devices for removing surgical bone cement from bone and, in particular, to a bone cement extractor that sequentially removes segments of cement from bone.

### BACKGROUND OF THE DISCLOSURE

Orthopedic implants, such as femoral stems, are cemented to the bone to maintain stability of the bone in connection with the human body. When these implants are removed, old cement remains in the cavity where the orthopedic implant was once located. Removal of the remaining cement is difficult and time consuming. Often, common techniques used to remove bone cement lead to damage to the bone and surrounding tissue. Also, cement fragments can be dislodged, causing inflammation, infection, and trauma to the patient.

### SUMMARY OF THE DISCLOSURE

According to an exemplary embodiment of the subject disclosure, there is provided a bone cement extractor for extracting cement from a bone cavity comprising a connection body and a segmented assembly. The connection body has a quick connect for connecting to a working tool. The segmented assembly is attachable to a distal end of the connection body and includes an inner segment having a proximal end and a distal end, the proximal end connectable to the distal end of the connection body, and an outer segment having a proximal end and a distal end, the outer segment slidably connectable to the inner segment.

According to an aspect, the inner segment and the outer segment interconnect by mechanically snap fitting. According to another aspect, the inner segment and the outer segment are movable relative to each other between an extended position and a contracted position.

According to an aspect, the bone cement extractor further includes an insert disposed within the inner segment for rigidifying the segmented assembly when the segmented assembly is in a contracted position. According to another aspect, the segmented assembly comprises a plurality of inner segments, and a plurality of outer segments arranged in alternating fashion with the plurality of inner segments. According to another aspect, a cap is connectable a distal end of a distalmost inner segment. According to another aspect, the cap has a substantially tubular body and a conical portion having a flange extending radially outwardly from the substantially tubular body about a distal end thereof.

According to an aspect, the inner segment has a substantially tubular body and the outer segment has a substantially tubular body, wherein the proximal end of the substantially tubular body of the outer segment includes a first radially inwardly extending lip, and the distal end of the substantially tubular body of the outer segment includes a second radially inwardly extending lip. According to another aspect, the outer segment includes a first annular recess and a second annular recess spaced from the first annular recess. According to another aspect, the outer segment further comprises a first countersink formed about an inner edge of its proximal end, and a second countersink formed about an inner edge of its distal end.

According to an aspect, the outer segment includes a radially outwardly extending rib. According to another aspect, the rib includes a substantially planar proximally facing end and a tapered distally facing end.

According to an aspect, the proximal end of the inner segment includes a plurality of spaced apart first snaps each having a first radially outwardly extending flange, and wherein the distal end of the inner segment includes a plurality of spaced apart second snaps each having a second radially outwardly extending flange. According to another aspect, the radially outwardly extending second flanges of the inner segment engage the first lip of a first outer segment, and the radially outwardly extending first flanges of the inner segment engage the second lip of another outer segment.

According to an aspect, the proximal end and the distal end of the inner segment are each threaded, the proximal end and the distal end of the outer segment are each threaded, and the threading of the inner segment is constructed and arranged to threadedly connect with and pass through the threading of the outer segment.

According to another exemplary embodiment of the subject disclosure, there is provided a method of removing bone cement from a bone comprising removing an implant from a bone canal, inserting new bone cement into the bone canal, inserting the aforementioned bone cement extractor into the bone canal, attaching a slide hammer to the bone cement extractor, expanding segments of the bone extractor by using the slide hammer to break and lift sections of bone cement, repeating operation of the slide hammer to break and pull out all sections of bone cement from the bone canal, and removing the bone cement extractor from the bone canal.

Other features and advantages of the subject disclosure will be apparent from the following more detail description of the exemplary embodiments.

### BRIEF DESCRIPTION OF THE SEVERAL VIEWS OF THE DRAWINGS

The following detailed description will be better understood when read in conjunction with the appended drawings. For the purpose of illustrating the subject disclosure, there are shown in the drawings exemplary embodiments. It should be understood, however, that the subject disclosure is not limited to the precise arrangements and instrumentalities shown.

FIG. 1A is a perspective view of a segmented cement extractor in accordance with an exemplary embodiment of the subject disclosure;

FIG. 1B is a perspective view of the segmented cement extractor of FIG. 1A attached to a slide hammer;

FIG. 1C is a perspective view of the segmented cement extractor of FIG. 1A attached to a T-handle;

FIG. 2A is a longitudinal cross-sectional elevation view of the segmented cement extractor of FIG. 1;

FIG. 2B is an enlarged, partial longitudinal cross-sectional elevation view of the segmented cement extractor of FIG. 1;

FIG. 3A is an enlarged perspective view of a quick connect of the segmented cement extractor of FIG. 1;

FIG. 3B is a longitudinal cross-sectional view of the quick connect of FIG. 3A;

FIG. 4A is an enlarged perspective view of an inner segment of the segmented cement extractor of FIG. 1;

FIG. 4B is an enlarged, longitudinal cross-sectional perspective view of the inner segment of FIG. 4A;

FIG. 4C is an enlarged side view of a first snap of the inner segment of FIG. 4A shown unflexed and in flexion;

FIG. 5A is a perspective view of an outer segment of the segmented cement extractor of FIG. 1;

FIG. 5B is a longitudinal cross-sectional perspective view of the outer segment of FIG. 5A;

FIG. 6A is a perspective view of a cap of the segmented cement extractor of FIG. 1;

FIG. 6B is a longitudinal, cross-sectional perspective view of the cap of FIG. 6A;

FIG. 7 is a longitudinal cross-sectional view of the segmented cement extractor of FIG. 1 connected to a sleeve;

FIG. 8 is a perspective view of a release mechanism within the sleeve of FIG. 7, with a casing of the sleeve removed for purposes of illustration;

FIG. 9 is a flowchart of a method of using the segmented cement extractor of FIG. 1;

FIG. 10A is a longitudinal view of the segmented cement extractor of FIG. 1 inserted into a cemented bone cavity shown in cross section;

FIG. 10B is a longitudinal view of the partially extended segmented cement extractor of FIG. 1 pulling out segments of cement from bone cavity shown in cross section; and

FIG. 11 is a longitudinal view of a portion of another embodiment of a segmented assembly of a segmented cement extractor according to the subject disclosure.

### DETAILED DESCRIPTION

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean towards the center of a body. The term “proximal” shall mean away from the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject application in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout the subject application, various aspects thereof can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the subject disclosure can be practiced without one or more of the specific features or advantages of a particular exemplary embodiment. In other instances, additional features and advantages may be recognized in certain embodiments that may not be present in all exemplary embodiments of the present disclosure.

A bone cement extractor 10 that removes cement from bone is disclosed. With the cement extractor, cement may be safely and quickly removed, while minimizing damage to the bone or trauma to the surrounding tissue.

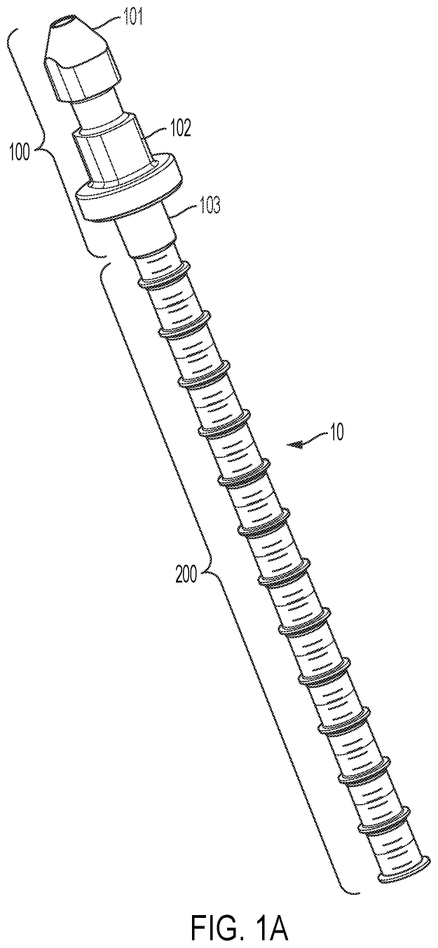

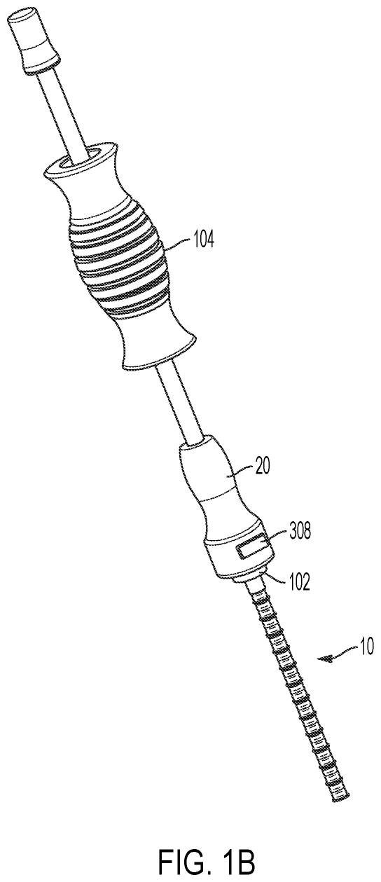

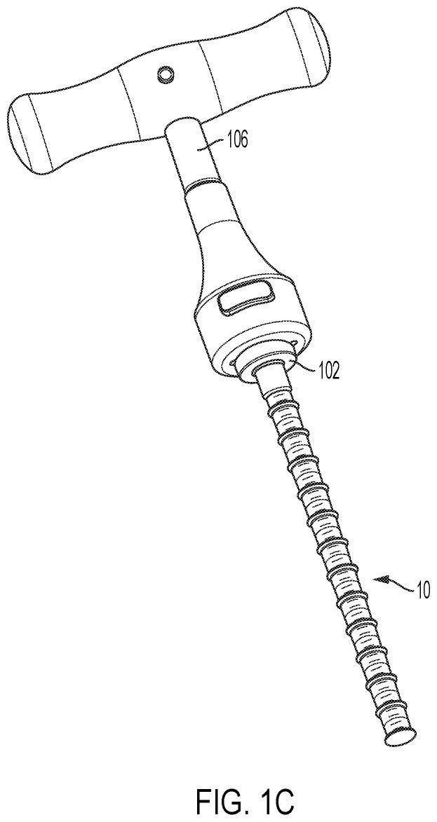

FIGS. 1A, 1B and 1C illustrate the bone cement extractor 10 in accordance with an exemplary embodiment of the subject disclosure. The bone cement extractor 10 includes a connection body 100 and a segmented assembly 200.

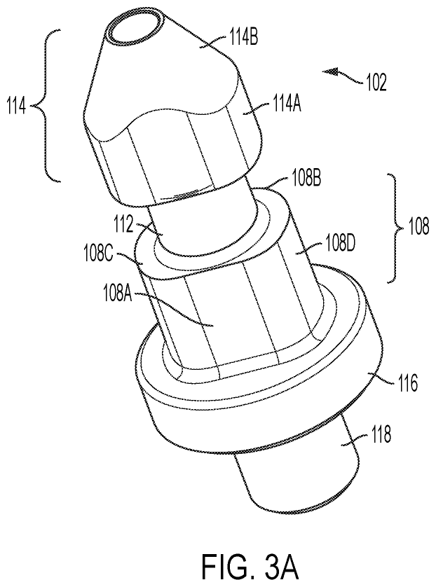

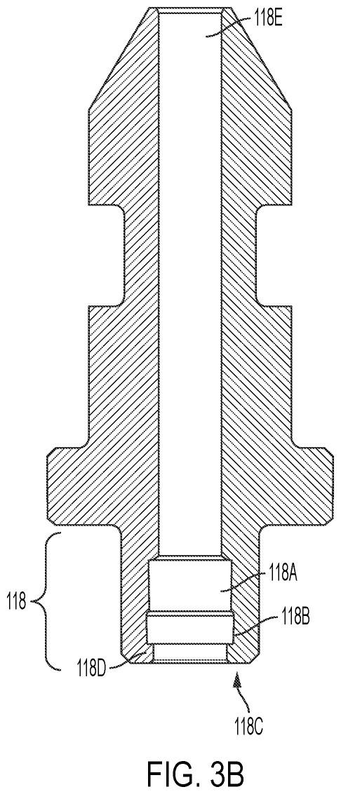

Referring to FIG. 3A, the connection body 100 has a proximal end 101 and a distal end 103, wherein the proximal end includes a quick connect 102, described in greater detail herebelow, for connecting to a working tool such as a slide hammer 104 (FIG. 1B), a T-handle 106 (FIG. 1C) or any other tool suitable for its intended purpose. The quick connect 102, is configured as best shown in FIGS. 3A and 3B and has a base section 108, a recessed section 112, a head section 114, a flange 116, and a receiving shaft 118. The base section 108 includes a pair of oppositely directed flats 108A, 108B each adjoined to a pair of oppositely directed rounds 108C, 108D. In an exemplary aspect, the base section is configured to have an oblong or oval transverse cross-sectional shape. The opposing flats and opposing rounds form the lateral walls of the base section.

The recessed section 112 extends from a first, proximal end of the base section 108 and is substantially cylindrical. The recessed section 112 also has a longitudinal cross-sectional area that is less than a longitudinal cross-sectional area of the base section, to allow for elements of the working tool to latch onto the recessed section and connect with the connection body 100.

The head section 114 extends from the recessed section 112 opposite to the base section 108 and includes a distal base end 114A and a substantially frustroconical proximal end 114B opposite the base end 114A. The base end 114A has a transverse cross-sectional profile the same as or substantially the same as the transverse cross-sectional profile of the base section 108. Further, the base end 114A has a larger transverse cross-sectional area than that of the transverse cross-sectional area of the recessed section 112, so that the working tool may latch on more securely to the connection body 100.

The flange 116 extends distally from the base section 108 at an end of the base section opposite the recessed section 112. The flange 116 has a circular profile. That is, the flange 116 has a circular transverse cross-sectional shape. The flange 116 also has a larger overall profile than that of the base section, so that the distal end of the working tool may butt against the flange 116, when the working tool connects with the connection body 100. In other words, the flange 116 extends radially outwardly from the base section 108.

The receiving shaft 118 extends from the flange 116 on a distal side opposite from which the base section 108 extends from the flange 116. As best shown in FIG. 3B, the receiving shaft 118 is cylindrical, and has a hollow interior 118A with an annular recess 118B adjacent a distal end 118C of the receiving shaft 118. A lip 118D extends radially inwardly from the annular recess 118B. The annular recess 118B is shaped to receive the segmented assembly 200, as will be explained in more detail below.

The quick connect 102 may also include a through hole 118E extending therethrough. The through hole 118E may extend co-linearly with a longitudinal central axis of the quick connect.

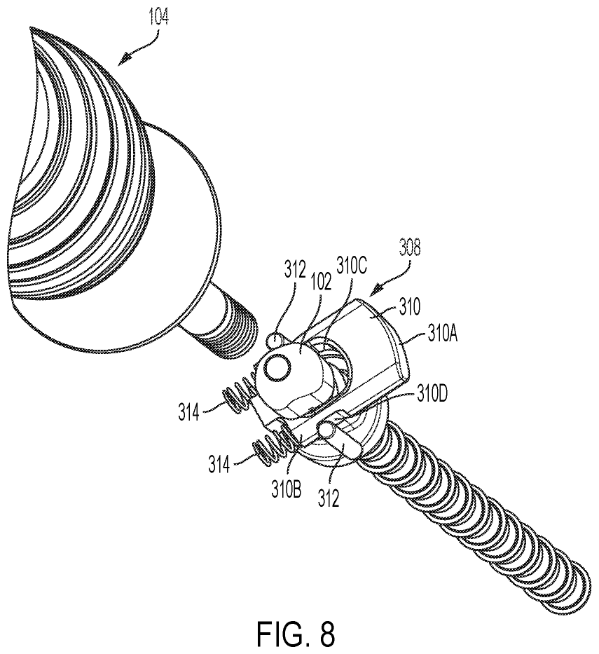

The bone cement extractor 10 is attached to the working tool such as the slide hammer 104 via a sleeve 20, as illustrated by FIGS. 1B, 7 and 8. The sleeve 20 has a proximal end 304 and a distal end 302. The distal end 302 includes a first slot 306, and a release mechanism 308. The first slot 306 is shaped to receive the quick connect 102 of the bone cement extractor 10. As best shown by FIG. 8, the release mechanism 308 includes a depressible catch ring 310, pins 312, and springs or other biasing members 314.

Referring to FIG. 8, the catch ring 310 includes a release button side 310A, a spring engagement side 310B, a slot 310C, and grooves 310D. The release button side 310A allows the user to push or depress the catch ring 310 to release the bone cement extractor 10 from being connected to the sleeve 20. Opposite the release button side 310A is the spring engagement side 310B, which engages the springs 314. The slot 310C allows the head 114 and the recessed section 112 of the quick connect 102 to pass through. The grooves 310D are located at opposite ends of the midportion of the catch ring 310.

The pins 312 fit on opposite ends surrounding the catch ring 310 and engage with the grooves 310D of the catch ring's midportion to allow the catch ring to move in a lateral direction relative to the longitudinal axis of the sleeve 20.

The springs 314, which may be in a pair, engage the spring engagement side 310B of the catch ring 310, and mechanically urge the catch ring 310 to engage the recessed section 112 of the quick connect 102 during connection of the quick connect 102 to the sleeve 20. Upon pushing on the release button side 310A of the catch ring 310 by a user's finger or thumb, the quick connect 102 along with the bone cement extractor 10 may disengage from the catch ring and the sleeve 20.

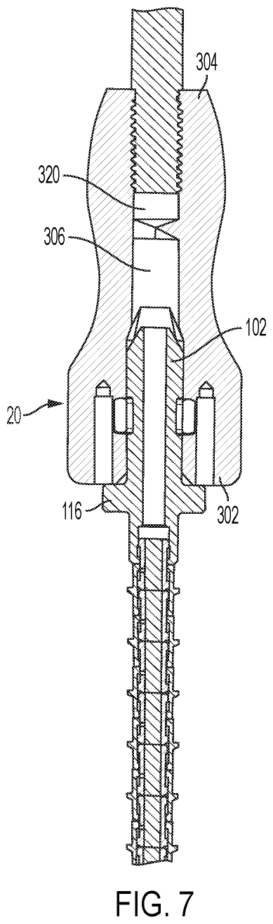

As best illustrated in FIG. 7, the proximal end of the sleeve 20 includes a second slot 320. The slot 320 extends through the sleeve 20 substantially coaxial with a longitudinal axis of the sleeve. The second slot 320 is shaped to receive the working tool, and may be threaded.

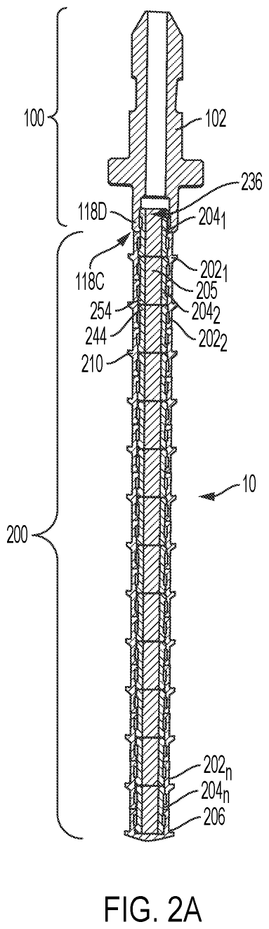

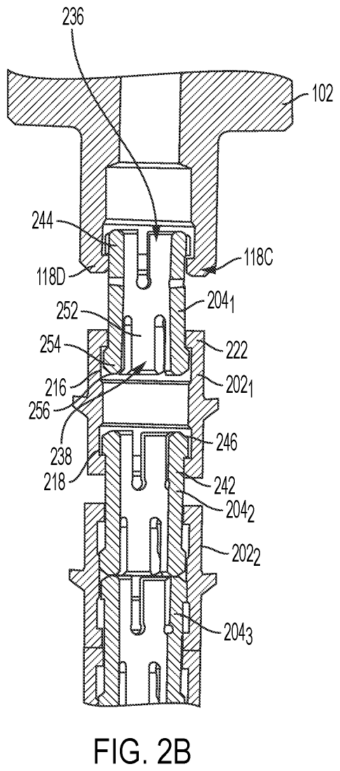

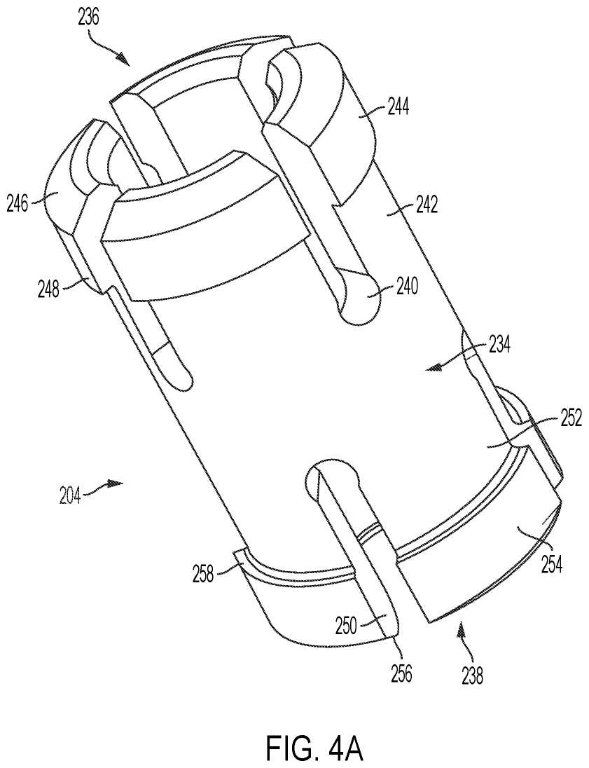

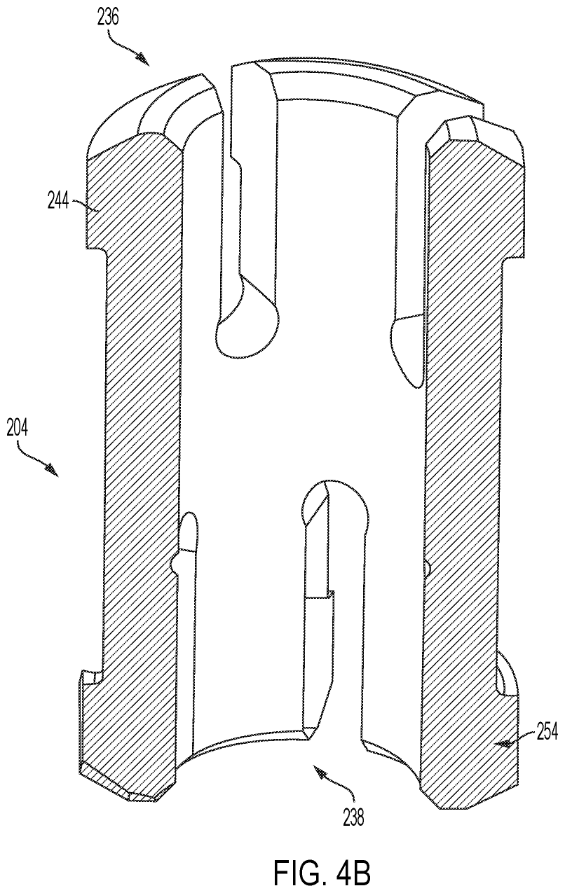

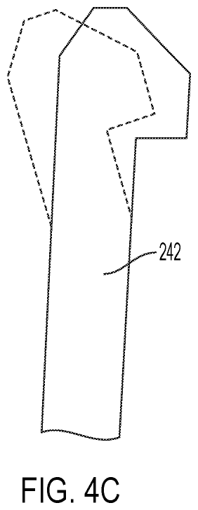

Referring to FIGS. 2A and 2B, the segmented assembly 200 is configured to extract cement from a bone cavity and includes an outer segment 202, or a plurality of “n” outer segments 202, an inner segment 204, or a plurality of “n” inner segments 204, an insert 205, and a distal cap 206. The inner segment 204, described in greater detail below, has a proximal end 236 and a distal end 238 (FIG. 4A), wherein the proximal end 236 is connectable to the distal end of the connection body 100. The outer segment 202, described in greater detail below, is configured to be slidably connectable to the distal end of the inner segment 204.

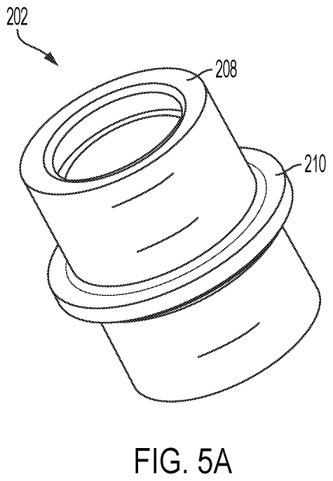

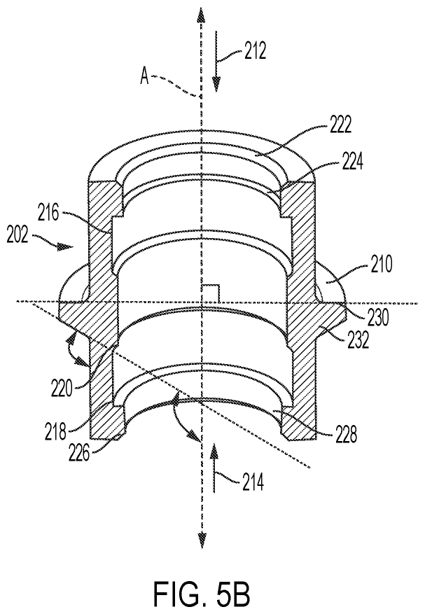

Each of the plurality of outer segments 202 is configured as best illustrated by FIGS. 5A and 5B. The outer segment 202 has a substantially tubular body 208 and a rib 210 extending radially outwardly about a midsection or midportion of the tubular body. The tubular body 208 has a proximal end 212, a distal end 214, a first annular recess 216 adjacent the proximal end, a second annular recess 218 adjacent the distal end, and a central passageway 220 extending through the tubular body.

Referring to FIG. 5B, the proximal end 212 includes a first countersink 222 and a radially inwardly extending first lip 224. The first countersink 222 is formed about an inner edge of the proximal end 212. The first lip 224 has an inner diameter that is less than an inner diameter of the first annular recess 216. In other words, the first annular recess 216 has an inner diameter that is greater than an inner diameter of the first lip 224.

The distal end 214 includes a second countersink 226 and a radially inwardly extending second lip 228. The second countersink 226 is formed about an inner edge of the distal end. The second lip 228 has an inner diameter that is less than an inner diameter of the second annular recess 218. In other words, the second annular recess 218 has an inner diameter that is greater than an inner diameter of the second lip 228. The first lip 224 may be sized to have the same inner diameter as the second lip 228, but can be configured to have a size smaller or greater than the second lip.

The central passageway 220 is in communication with the first annular recess 216 on one end of the central passageway, and with the second annular recess 218 on the other end of the central passageway. The central passageway has an inner diameter less than those of the first annular recess 216 and the second annular recess 218, but more than those of the first lip 224 and the second lip 228.

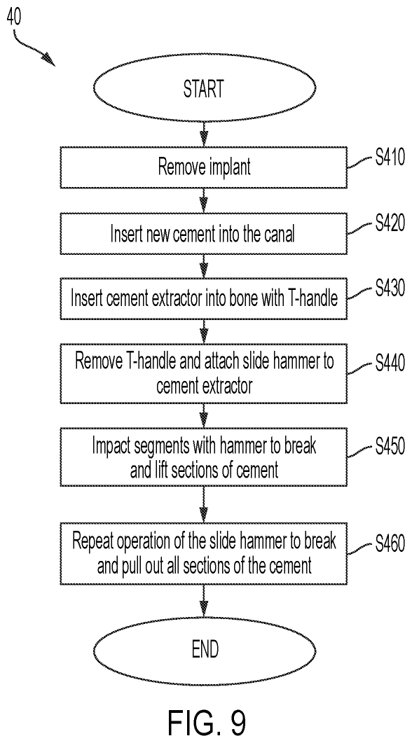

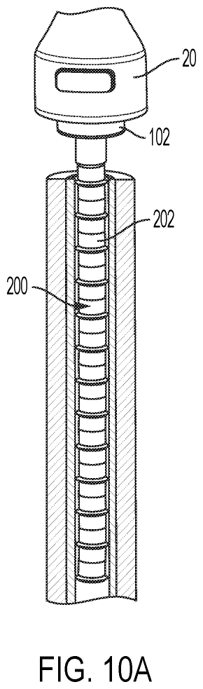

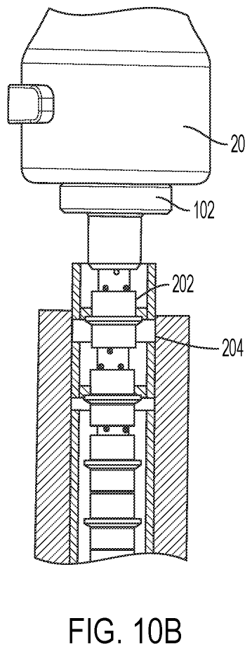

The rib 210 circumscribes the substantially tubular body 208 about a midportion thereof. The rib 210 includes a substantially planar proximally facing end 230 and a tapered distally facing end 232. The substantially planar proximally facing end 230 is substantially perpendicular to the longitudinal axis of the tubular body 208. This allows for the rib 210 to engage inwardly directed bone cement, break off the cement, and pull out the broken pieces or sections of the cement during the extraction of the segmented assembly 200 when in use. The tapered distally facing end 232 of the rib 210 is angled, e.g., greater than 90 degrees (i.e., relative to the longitudinal axis “A” of the tubular body 208). This allows for ease of insertion of the segmented assembly 200 into the bone cavity, and for removing cement debris from the bone cavity. Both the insertion and the extraction of the segmented assembly 200 will be discussed in more detail with regard to FIGS. 9, 10A, and 10B.

The inner segment 204, as best shown by FIGS. 4A and 4B, includes a substantially tubular body 234. The proximal end 236 of the substantially tubular body 234 is divided by a plurality of first longitudinally extending slots 240 forming first snaps 242, each having a first radially outwardly extending flange 244. That is, the proximal end includes a plurality of spaced apart first snaps 242.

Each of the first longitudinally extending slots 240 extend from the proximal end 236 towards a center section of the substantially tubular body 234. Also, the first snaps 242, which are formed by the first longitudinally extending slots 240, extend from about or adjacent the center section of the substantially tubular body 234 towards the proximal end 236. The first snaps 242 are radially flexible and movable radially between an unflexed first position having a first overall diameter, and a flexed second position having a second overall diameter (see FIG. 4C). When in the first position, the first overall diameter formed by the plurality of first snaps is larger than the second overall diameter in the second or flexed position. Further, the first radially extending flange 244 extends radially outwardly from the first snaps 242 and has a conical or chamfered first entrance side 246 facing the proximal end 236 and a squared off first exit side (i.e., first overhang or shoulder) 248 opposite the entrance side. The first overhang 248 can have a depth of about 0.1, 0.5, 1.0, 1.5, 2, 3, 4, 5, 6, 7, 8, 9, 10, 12, 14, 16, 18, 20, 25, 30, 35, 40 mm, or more.

The distal end 238 of the substantially tubular body 234 is divided by a plurality of second longitudinally extending slots 250 defining second snaps 252, each having a second radially outwardly extending flange 254. That is, the distal end includes a plurality of spaced apart second snaps 252.

Each of the second longitudinally extending slots 250 extend from the distal end 238 towards the center section of the substantially tubular body 234. Also, the second snaps 252, which are defined by the second longitudinally extending slots 250, extend from about or adjacent the center section of the substantially tubular body 234 towards the distal end 238. The second snaps 252 are radially flexible and movable radially between an unflexed first position having a first overall diameter, and a flexed second position having a second overall diameter. (Similar to the first snaps 242 as depicted in FIG. 4C). When in the first position, the first overall diameter formed by the plurality of second snaps is larger than the second overall diameter in the second or flexed position. Further, the second radially extending flange 254 extends radially outwardly from the second snaps 252 and has a conical or chamfered second entrance side 256 facing the distal end 238 and a squared off second exit side (i.e., second overhang or shoulder) 258 opposite the entrance side. The second overhang 258 can have a depth that is substantially the same as the depth of the first overhang 248.

Referring again to FIG. 2A, the insert 205 is a cylinder that fits within the inner segment 204. The insert 205 has about the same length as that of the inner segment 204 and has about the same diameter as the inner diameter of the tubular body 234 of the inner segment 204 but sufficient to allow the insert to be received within the inner segment. Alternatively, the insert can have a length lesser than or greater than the length of the inner segment.

Referring also to FIG. 2B, the first inner segment 204 of the segmented assembly 200 connects to the connection body 100 by a snap-fit mechanism through the distal end 118C of the quick connect 102 (in which the proximal end 236 of the first inner segment 204 fits through the lip 118D of the quick connect 102).

According to a first exemplary embodiment of the subject disclosure, the outer segments 202 and the inner segments 204 easily and quickly interconnect by mechanically snapping together. For example, referring to FIG. 2B, the interconnection between the first inner segment 2041 with the first outer segment 2021 is achieved by having the second entrance side 256 of the second snaps 252 at the distal end 238 of the first inner segment 2041 engage the first countersink 222 at the proximal end 212 of the outer segment 202. The second snaps 252 along with the second radially extended flanges 254 flexes radially inward as the inner segment 204 is inserted into the inside of the outer segment 202, and the second radially extended flange 254 fits through the first countersink 222 of the first outer segment 2021. When the second radially outwardly extended flange 254 fits entirely through the first countersink 222 of the first outer segment 2021 (i.e., second first overhang 258 passes through the first countersink 222), the second snaps 252 flex back to their original shape, and the second radially extended flanges 254 on the distal end 238 of the inner segment 204 extends within the first annular recess 216 of the first outer segment 2021. The inner segment 204 is now interlocked with the outer segment 202. Once interlocked, the insert 205 may then be inserted within the cavity of the inner segment 204 to further bend the inner segment 204 back into its original shape, and provide support for the overall structure, as shown in FIG. 2A. The same may be repeated to interlink the distal end 238 of successive inner segments 204 with the proximal end 212 of successive outer segments 202 so that the distal end 238 of each inner segment 204 is interlinked with each respective outer segment 202.

Referring again to FIG. 2B, once connected, the outer segments 202 and the inner segments 204 are slidably movable relative to each other between an extended position and a contracted position. As best exemplified by FIG. 2B, while in the extended position, the second radially outwardly extended flanges 254 of the inner segments 2041 engages the first lip 224 of the outer segment 202 at the second overhang 258 of the first inner segment 2041. Also, the second lip 228 of the outer segment 202 engages with the first radially outwardly extended flanges 244 of another (e.g., second segment 2042) at the second overhang 258 of the other inner segment 204.

As also shown in FIG. 2B, while in the contracted position, the distal end 238 of the inner segment 204 (e.g., second segment 2042) abuts the proximal end 236 of another inner segment 204 (e.g., 2043). In an embodiment, the insert 205 is also disposed within the inner segment 204, as seen in FIG. 2A. The contact of the second and first radially extended flanges 254, 244 of the inner segments 204 with the central passageway 220 of the outer segment 202, and the placement of the insert 205 within the inner cavity of the above-described elements, rigidifies the entire segmented assembly 200 while in the contracted position, e.g., so as to prevent the flexing of the overall segmented assembly.

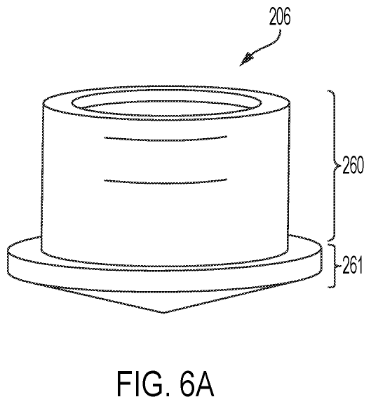

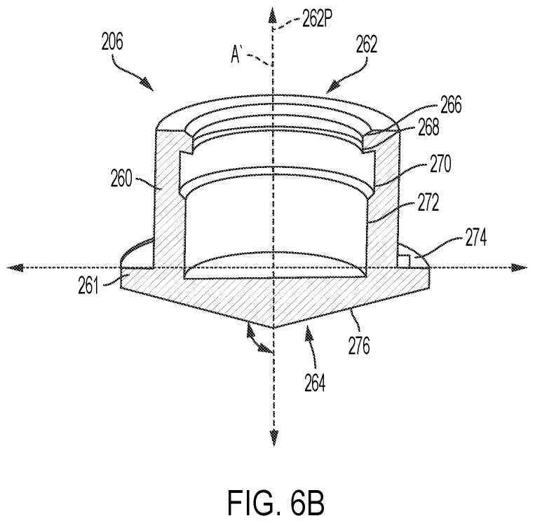

Referring to FIGS. 2A, 6A and 6B, the cap 206 fits on the distal end of the segmented assembly 200 opposite where the segmented assembly 200 connects with the connection body 100. The cap 206 has a substantially tubular body 260 at a proximal end 262 and a conical portion 261 at a distal end 264. The proximal end 262 includes a countersink 266 and a lip 268. The countersink 266 extends from the proximal end 262 towards an interior of the cap 206 and has an annular shape. The countersink has a depth of about ½ of the overall length of an inner segment 204n so as to accept and retain a portion of the inner segment 204n, as shown (e.g., in FIG. 2A). A lip 268 extends from the countersink 266 and has a diameter that is equal to or less than that of the countersink 266.

An annular recess 270 is adjacent the lip 268 about the proximal end 262 of the tubular body 260. The annular recess 270 has an inner diameter that is greater than an inner diameter of the lip 268. An annular wall 272 is distal to the recess 216. The annular wall 272 has an inner diameter less than that of the annular recess 270, but greater than that of the lip 268.

The conical portion 261 circumscribes and extends from the substantially tubular body 260 about a distal end thereof. The conical portion 261 includes a substantially planar proximally facing end 274 and a tapered distally facing end 276. The substantially planar proximally facing end 274 is substantially perpendicular to a longitudinal axis “A” of the tubular body 260. This allows for the conical portion 261 to engage inwardly directed bone cement, break off the cement, and pull out the lowest or distalmost broken pieces or sections of cement reachable by the segmented assembly 200 during extraction thereof. In other words, the cap includes a flange extending radially outwardly from the tubular body. The tapered distally facing end 276 is angled, e.g., greater than 90 degrees (i.e., relative to the longitudinal axis A′ of the tubular body 260). This allows for ease of insertion of the segmented assembly 200 into the bone cavity, and of removing of cement debris from the bone cavity.

The cap 206 interlocks with the last or distalmost inner segment 204n opposite the first inner segment 2041 connected to the connection body 100, thereby encapsulating the distal end 238 of the last inner segment 204n.

In operation, as best illustrated by the flowchart of FIG. 9 and FIGS. 10A and 10B, a method 40 of removing cement from the bone, for example, from a femoral canal, is described. At S410, an implant, such as a femoral stem in the canal of the femur, is removed from the bone. Then, new bone cement is inserted into the canal at S420 to bond to the existing cement in the canal. Once the new bone cement is inserted into the canal, and before the new cement hardens, at S430 the bone cement extractor 10 is inserted into the bone cement (see also FIG. 10A). This is done by connecting the bone cement extractor 10 with a working tool, such as a t-handle, which may be used to insert the bone cement extractor 10 into the new bone cement. Here, the sections of the inner segments 204 and outer segments 202 of the bone cement extractor 10 are pressed together in order to keep the bone cement extractor 10 rigid while being inserted. Also, the tapered distally facing end 232 of the ribs 210 on the outer segments 202 of the bone cement extractor 10 facilitates its insertion into the new cement.

Once the bone cement hardens, at S440 the t-handle is removed from the bone cement extractor 10, and an impacting tool, such as a slide hammer 104 can be attached to the bone cement extractor 10. Next, at S450, the slide hammer 104 is used to impact sections of inner segments 204 and outer segment 202, causing the cement in the bone to break into small pieces as the inner and outer segments move from the contracted position to the extended position, described above (see also FIG. 10B). That is, the weight of the slide hammer 104 is moved along its shaft to strike the stop at the end of the slide hammer 104 opposite the end connected to the cement extractor 10, the impact followed by the force of the slide hammer 104 separates the sections of the inner segments 204 and the outer segment 202, and causing the planar proximally facing ends 230 of the outer segments 202 to engage and break the cement into layers along the ribs 210 of the outer segments. This method of successive layers being broken apart by successive segments of the segmented assembly advantageously allows for the breakage and removal of bone cement while applying less overall forces to the bone, thereby imparting less damage to the bone.

After breaking the cement into layers, at S460, cyclic pulling force is generated by the slide hammer as its weight is repeatedly moved back and forth along the shaft of the slide hammer 104. This generates successive pulling or extraction forces that incrementally pull the sections of the inner segments 204 and the outer segments 202 out of the bone cavity. That is, the sections of the inner segments 204 are moved upward out of the bone, taking with them the broken layers of the cement from the bone along with each outer segment 202. Here, the interconnectedness of the inner segments 204 and outer segments 202 keep the segments of the cement extractor 10 connected while successive layers of the cement are broken and pulled out. The operation of the slide hammer at S460 continues until all of the cement has been broken into layers by the impact force of the slide hammer 104 and pulled out.

The bone cement extractor and its use allow for a more complete removal of the remaining cement in the bone cavity, after the removal of bone implants, while minimizing damage to the bone and surrounding tissue. That is, the breakage of the cement into pieces or sections requires less force to be applied to dislodge and take out all of the cement from the bone cavity. Also, the interconnectedness between the inner segments 204 and the outer segments 202 keeps all of the elements of the bone cement extractor 10 together, as the elements of the bone cement extractor 10 work together to break and pull out the layers of the cement. That is, all of the segments of the bone cement extractor 10 stay operatively connected to each other during use. Such a construction and arrangement prevent a user from having to separately thread a cement extraction segment into each segment connected to the cement as the assembly is inserted into the bone cavity. This greatly reduces the time needed to remove the bone cement and simplifies the process, while preventing pieces of the bone cement extractor 10 from being lost.

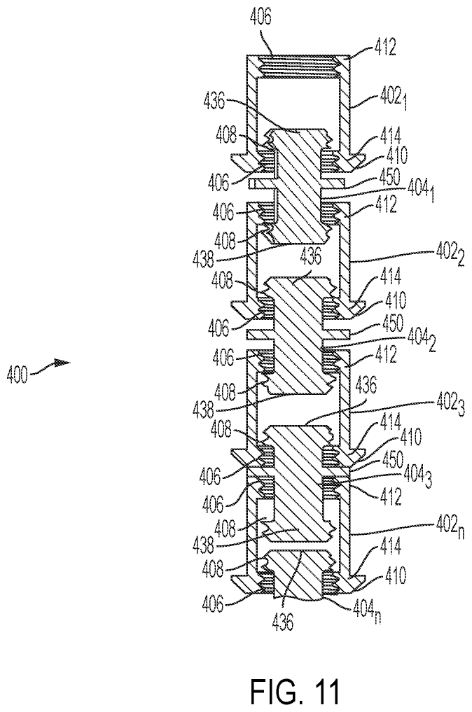

Referring to FIG. 11, there is shown a further embodiment of a segmented assembly 400 for use in the bone cement extractor according to the subject disclosure. Like the segmented assembly 200 described above, the segmented assembly 400 includes one or more inner segments 404, e.g., 4041 through 404n, and one or more outer segments 402, e.g., 4021 through 402n, that are connectable to one another.

Each outer segment 402 has a tubular body including a proximal end 412 and a distal end 414. Fasteners, such as internal threading 406 is provided at each of the proximal and distal ends 412, 414. A radially outwardly extending rib 410, similar in construction and function to rib 210, described above, is provided on the exterior of the tubular body. The outwardly extending rib 410 can be positioned about the proximal end 412, a midportion or, as illustrated, adjacent the distal end 414.

Each inner segment 404 has a substantially cylindrical body including a proximal end 436 and a distal end 438. External threading 408 is provided at each of the proximal and distal ends 436, 438. Each inner segment can include a radially outwardly extending flange 450 about a midportion thereof for facilitating screwing of an inner segment 404 into an outer segment 402. According to an aspect, a diameter of the flange 450 is less than a diameter of the rib 410 of the outer segment(s) 402.

Although not illustrated, it will be understood that the distal end of the above-described sleeve 20 can be provided with external threading constructed and arranged for firm seating engagement of a proximal outer segment, e.g., outer segment 4021, with the sleeve. Conversely, it will be understood that the distal end of the above-described sleeve 20 can be provided with internal threading constructed and arranged for firm seating engagement of a proximal inner segment, e.g., inner segment 4041, with the sleeve.

Similarly, segmented assembly 400 may be provided with a distal cap similar to the above-described distal cap 206 disposed at a distal end of the segmented assembly 400. It will be likewise understood that such a distal cap can be provided with external threading constructed and arranged for firm seating engagement of a distal outer segment, e.g., outer segment 402n, with the distal cap. Conversely, it will be understood that the distal end of the distal cap can be provided with internal threading constructed and arranged for firm seating engagement of a distal inner segment, e.g., inner segment 404n, with the distal cap.

According to this embodiment, the inner and outer segment(s) 404, 402 are interconnected by inner and outer mating threading 406, 408 on the respective outer and inner segments that is initially engaged with one another and then entirely passed by one another. That is, when installing successive inner and outer mating segments 404, 402 into each other, the cooperating threading 406, 408 is initially engaged by screwing either an inner segment 404 into an outer segment, or an outer segment around an inner segment. Screwing of the respective inner or outer segment continues until the respective threadings of the inner and outer segments are no longer engaged. In other words, once their respective threadings fully pass each other, the inner and outer segments become loosely interlocked in a slidable relationship, whereby the ends of the inner segments become trapped in the outer segments such that the threads of the inner and outer segments abut one another to prevent separation of the inner and outer segments in tension.

It will be appreciated by those skilled in the art that changes could be made to the exemplary embodiments described above without departing from the broad inventive concept thereof. It is to be understood, therefore, that this disclosure is not limited to the particular embodiments disclosed, but it is intended to cover modifications within the spirit and scope of the subject disclosure as defined by the appended claims.

1. A bone cement extractor for extracting cement from a bone cavity comprising:
a connection body having a quick connect for connecting to a working tool; and
a segmented assembly attachable to a distal end of the connection body, the segmented assembly including:
an inner segment having a proximal end and a distal end, the proximal end connectable to the distal end of the connection body, and
an outer segment having a proximal end and a distal end, the outer segment slidably connectable to the inner segment.

2. The bone cement extractor of claim 1, wherein the inner segment and the outer segment interconnect by mechanically snap fitting.

3. The bone cement extractor of claim 1, wherein the inner segment and the outer segment are movable relative to each other between an extended position and a contracted position.

4. The bone cement extractor of claim 1, further comprising an insert disposed within the inner segment for rigidifying the segmented assembly when the segmented assembly is in a contracted position.

5. The bone cement extractor of claim 1, wherein the segmented assembly comprises a plurality of inner segments, and a plurality of outer segments arranged in alternating fashion with the plurality of inner segments.

6. The bone cement extractor of claim 1, further comprising a cap connectable to a distal end of a distalmost inner segment.

7. The bone cement extractor of claim 6, wherein the cap has a substantially tubular body and a conical portion having a flange extending radially outwardly from the substantially tubular body about a distal end thereof.

8. The bone cement extractor of claim 1, wherein the inner segment has a substantially tubular body and the outer segment has a substantially tubular body, and wherein the proximal end of the substantially tubular body of the outer segment includes a first radially inwardly extending lip, and the distal end of the substantially tubular body of the outer segment includes a second radially inwardly extending lip.

9. The bone cement extractor of claim 1, wherein the outer segment includes a first annular recess and a second annular recess spaced from the first annular recess.

10. The bone cement extractor of claim 1, wherein the outer segment further comprises a first countersink formed about an inner edge of its proximal end, and a second countersink formed about an inner edge of its distal end.

11. The bone cement extractor of claim 1, wherein the outer segment includes a radially outwardly extending rib.

12. The bone cement extractor of claim 11, wherein the rib includes a substantially planar proximally facing end and a tapered distally facing end.

13. The bone cement extractor of claim 10, wherein the proximal end of the inner segment includes a plurality of spaced apart first snaps each having a first radially outwardly extending flange, and wherein the distal end of the inner segment includes a plurality of spaced apart second snaps each having a second radially outwardly extending flange.

14. The bone cement extractor of claim 13, wherein the radially outwardly extending second flanges of the inner segment engage the first lip of a first outer segment, and the radially outwardly extending first flanges of the inner segment engage the second lip of another outer segment.

15. The bone cement extractor of claim 1, wherein the proximal end and the distal end of the inner segment are each threaded, wherein the proximal end and the distal end of the outer segment are each threaded, and wherein the threading of the inner segment is constructed and arranged to threadedly engage with and pass through the threading of the outer segment.

16. A method of removing bone cement from a bone comprising:
removing an implant from a bone canal;
inserting new bone cement into the bone canal;
inserting the bone cement extractor of claim 1 into the bone canal;
attaching a slide hammer to the bone cement extractor;
expanding segments of the bone extractor by using the slide hammer to break and lift sections of bone cement;
repeating operation of the slide hammer to break and pull out all sections of bone cement from the bone canal; and
removing the bone cement extractor from the bone canal.
